(19) United States
(12) Patent Application Publication
(10) Pub. No.: US 2024/0148581 A1
(43) Pub. Date: May 9, 2024

(54) PATIENT TABLE MOUNTING FRAME

(71) Applicant: Brainlab AG, Munich (DE)

(72) Inventors: Stefan HOFBERGER, Munich (DE); Norman PLASSKY, Munich (DE)

(21) Appl. No.: 18/279,344

(22) PCT Filed: Mar. 24, 2021
(86) PCT No.: PCT/EP2021/057567

§ 371 Date: Aug. 29, 2023

(51) Int. Cl. A61G 13/10; A61B 34/32

(52) U.S. Cl. CPC A61G 13/101; A61B 34/32; A61B 2090/571

(57) ABSTRACT

A patient table mounting frame includes a first frame part and a second frame part coupled to each other via a translatory guide that enables the first frame part and the second frame part to move with respect to each other in a first direction, wherein the first frame part and the second frame part each comprise a table engagement section adapted to releasably connect to a patient table or parts thereof, and wherein at least one of the first frame part and the second frame part includes a mounting section adapted to releasably connect to a corresponding associated mounting adaptor.

### FIELD OF THE INVENTION

The present invention relates to mounting systems for attaching medical appliances such as medical robotic systems or medical articulated support arms to a patient table that a patient rests on during a medical procedure.

### TECHNICAL BACKGROUND

Robotic assistance plays an increasingly important role in the field of surgery as it improves the capabilities of surgeons, particularly when it comes to high precision surgery and procedures performed on hard to reach parts of a patient's body. Commonly used robotic systems are often provided as standalone systems mounted on a cart for placing the robotic system at a desired position with respect to the patient. In order to ensure that the robotic system remains unaffected from any unintended outside influence such as medical personnel bumping against the cart, those carts are built as sturdy structures which are therefore heavy, cumbersome and require a considerable amount of space that could be used otherwise by medical personnel. Thus, there is an ongoing need for systems to have a small form factor and to integrate into medical procedures more tolerably. Systems are know which connect between a robot and a patient table and therefore exhibit a small footprint. These systems are however at the same time confronted with the problem that widely used interfaces provided by conventional patient tables, such as side-rails of the patient table, are not specifically designed for supporting medical robots and for taking up loads applied by those robots. EP 3 644 890 A1 suggests a head-holder support to be mounted to a patient table, i.e. to side-rails which extend laterally to the patient table, and further includes dedicated sections for receiving a robotic system. The suggested head-holder support overcomes the drawback of previous, space-consuming systems.

It is the object of the present invention to provide a versatile, low-footprint, but still highly robust system for installing a medical robot or similar structure within a medical environment. In particular, it is the object of the present invention to provide a system which is, on the one hand, capable of being mounted to various types of patient tables, and, on the other hand, provides much leeway for installing a medical robot or similar structure at a desired position with respect to the patient.

The present invention can be used for in connection with any system for robot-assisted medical procedures, such as Cirq®, a product of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

The present invention relates to a patient table mounting frame comprising a first frame part and a second frame part coupled to each other via a translatory guide that enables the first frame part and the second frame part to move with respect to each other in a first direction, wherein the first frame part and the second frame part each comprise a table engagement section adapted to releasably connect to a patient table or parts thereof, and wherein at least one of the first frame part and the second frame part includes a mounting section adapted to releasably connect to a corresponding mounting adaptor.

### GENERAL DESCRIPTION OF THE INVENTION

In this section, a description of the general features of the present invention is given for example by referring to possible embodiments of the invention.

The present invention addresses the aforementioned objects by providing a patient table mounting frame that comprises a first and a second frame part which are coupled to each other via a preferably lockable translatory guide that enable the first part and the second part to move with respect to each other in a first direction. The first frame part and the second frame part each comprise a table engagement section that is adapted to releasably connect to a patient table, wherein at least one of the first frame part and the second frame part includes a mounting section adapted to releasably connect to a correspondingly formed mounting adaptor configured to support a medical robot or similar device.

In other words, the inventive mounting frame is basically built as a two-part-structure adapted to receive a patient table between these two frame parts. The two frame parts may engage the patient table or parts thereof from opposite directions, particularly by being moved towards each other along the translatory guide. While both of the two frame parts connect to the patient table, at least one of the frame parts is also adapted to connect to a mounting adaptor which, in some embodiments, does not form part of the present invention. Rather, the mounting adaptor may be provided as an intermediate structure that directly connects to a frame part of the inventive mounting frame on the one hand, and to a surgical robot or similar support structure on the other hand. Further, the mounting adaptor may also be provided as integrated part of the robot or support structure.

Thus, with any configuration of the inventive mounting frame, a first one of the two frame parts may serve as an intermediate structure between the patient table and a surgical robot, that, apart from fastening means, directly connects between the patient table and the robot so as to mount the robot to the patient's table in a most rigid manner. As the number of elements between the table and the robot is thereby reduced to the absolute minimum without any hinges or connecting interfaces between different frame sections that come along with tolerances and plays, the present invention ensures that the spatial position of a robot is accurately maintained with respect to the patient table.

Further, and in any configuration, the second of the two frame parts may serve as an intermediate structure which, apart from fastening means, directly connects between the first frame part and the patient table or parts thereof, thereby reducing the number of structures between the first frame part and an additional connection to the patient table to an absolute minimum, as well.

All in all, the robot is connected to a first section of the patient table via one single frame part, and to a second section of the patient table via both of the frame parts. As the two frame parts can be displaced with respect to each other via the translatory guide, the inventive frame does not only feature a high rigidity, but may also engage various types of patient tables having different dimensions, particularly widths.

In one example, the frame, i.e. both parts thereof may be integrally formed elements and/or may include or be formed from suitable (e.g. radiolucent) material for allowing the frame to be used during various medical imaging procedures including X-Ray-, CT- and/or MR-imaging-procedures, e.g. carbon fibre reinforced plastic.

In a further example, those sections of the first frame part and the second frame part which engage the patient table extend in a direction which is transversal, particularly perpendicular to the direction in which the frame parts are movable with respect to each other.

Moreover, the table engagement sections of the respective frame parts may extend parallel to each other. This allows the mounting frame to engage a certain length of the table or side-rails thereof which extend laterally to the patient's table, wherein the mounting frame may be adapted to various widths of patient tables by moving the frame parts with respect to each other along the translatory guide.

While the mounting frame itself is designed as a most sturdy and rigid support structure, the patient table or parts thereof may be of less sturdiness. In order to get the most stability out of those structures, the inventive mounting frame may engage or connect to the patient table or parts thereof over a predefined distance, particularly over at least 10 cm specifically over at least 20 cm, more specifically over at least 30 cm. By doing so, load applied to the robot acts on large sections of the patient table or parts thereof, thereby reducing possible deformations as much as possible. One can easily imagine that a predefined amount of force or torque will cause less deformation when distributed over a large section of a solid object, as this would be the case when the same amount of force or torque is applied to a smaller section of that object. Thus, the present invention allows for a rigid connection between the patient table and the robot even if certain parts of the patient table are not specifically designed to serve as an interface for connecting robots or similar structures for high-precision medical procedures.

In order for the mounting frame to be connected to various types of patient tables, the table engagement sections of the first frame part and the second frame part may include one or more engagement members, or may connect to one or more engagement members, wherein the one or more engagement members are adapted to releasably engage at least one of
a side-rail of the patient table which particularly extends laterally of the tabletop of the patient table;
a frame of the patient table;
an auxiliary support plate adapted to rest on the upper surface of the tabletop of the patient table.

While the table engagement section of one or both of the frame parts may feature discrete attachment points for one or more engagement members, one or both of the engagement sections may also include sort of an engagement rail or similar structure which allows one or more of the engagement members to connect to a respective engagement section at any desired location along the engagement rail. Thus, positioning the one or more engagement members is not limited by any constraints on the mounting frame side, but may be freely chosen to find the best possible location for connecting to the patient table or parts thereof.

In a further example, the one or more engagement members may releasably connect to the patient table or parts thereof via a friction fit and/or a positive fit. For example, the engagement members may receive a side rail to only allow a translational degree of freedom along the length of the side rail. This remaining degree of freedom may be fixed by a clamping mechanism of the engagement members that establishes a friction fit between the side rail and the corresponding engagement member.

In a further example, the mounting frame includes a locking mechanism configures to selectively fix the position of the first frame part and the second frame part with respect to each other. While this can be done via a positive fit at discrete relative positions of the frame parts, a friction fit allows fixing the frame parts to each other in any desired relative position. The frictional force for maintaining the relative position between the frame parts may for example be significantly increased with the help of “wedge”- or V-shaped sections of the translatory guide, which are pressed against each other via the locking mechanism.

In a further example, the mounting frame comprises a drive mechanism configured to move the first frame part and the second frame part with respect to each other along the translatory guide. The drive mechanism may include one or more actuators or motors, but may also be manually activated, for example via a lever or crank or similar means allowing medical personnel to manually move the frame parts towards each other or away from each other. In this regard, the mounting frame, particularly the engagement sections may be configured to releasably connect to the patient table or parts thereof by being pressed against the patient table or parts thereof via the drive mechanism. In a specific example for connecting the mounting frame to a patient table, the two parts of the mounting frame may be positioned with respect to each other to have an opening for receiving the patient table, which is slightly wider than the actual width of the patient table. After the patient table is received between the two parts of the mounting frame, the drive mechanism may be activated to move both parts of the mounting frame towards each other so as to clamp the patient table therebetween.

In a further example, the first frame part and the second frame part of the mounting frame are adapted to connect to each other along the translatory guide with no intermediate structures therebetween. For example, one of the frame parts may include a cross-sectional extension which is received by a correspondingly shaped cross-section recess of the other one of the frame part. The extension and the recess may have a tapered cross-sectional shape which helps in increasing friction between the two frame parts when, as was previously remarked, the locking mechanism is applied.

While the inventive mounting frame is to provide a most rigid connection between the patient table and a robot or similar structure, the inventive mounting frame may further be formed to exhibit a slim shape that takes away as less space as possible. Thus, the support frame may have a C-shaped geometry with the table engagement sections of the first frame part and the second frame part being spaced apart and extending substantially parallel to each other, wherein the translatory guide extends between respective ends of the engagement sections. This C-shaped geometry allows for positioning the entire support frame underneath a patient table, wherein the table column may be received between the engagement section.

Further, the overall cross-sectional shape of the support frame as seen along a longitudinal axis of the frame and the table may taper in a downward direction. It is then possible for the mounting frame to remain connected to a patient table even when the table is received within a tube shaped opening of a medical imaging device such as an MRI-device.

In a further example, the mounting frame may include one or more mounting adaptors which are to connect to the mounting section of either one of the first frame part and the second frame part. In such example, the mounting adaptor does form part of the inventive mounting frame.

For example, the mounting section may therefore include an engagement rail that enables the one or more mounting adaptors to connect to a respective mounting section at any desired location along the engagement rail. Just like the engagement rails provided for the engagement members that connect to the patient table or parts thereof, the engagement rails provided for the at least one mounting adaptor therefore allow for great flexibility in positioning medical appliances such as a robotic system with respect to the patient table. For example, a robotic system may not only be mounted to the patient table at any desired lateral position along the mounting frame, but may also be mounted either left or right to the patient table, thereby leaving great flexibility for robot-assisted procedures.

In further examples, at least one mounting adaptor is to connect a respective robotic system to the mounting frame. The adaptor may therefore be configured to form part of the robotic system but may also be configured to releasably connect to such robotic system as an intermediate structure between the robotic system and the mounting frame.

Further, it is conceivable to provide at least one mounting adaptor that is formed as a rail extending laterally to and at a distance from one of the table engagement sections. Thus, an auxiliary structure is provided for attaching medical appliances which would normally be attached to the table side-rails which are now occupied by the mounting frame.

In a further example, the mounting frame may further comprise a head-clamp mounting section which is adapted to releasably connect to at least one of the first frame part and the second frame part. The head-clamp mounting section may connect to at least one of the frame parts at any expedient location, for example at a location lateral to a frame part, a location underneath a frame part, a location above a frame part, or a location at the front of a frame part. In particular, the head-clamp mounting section may connect to at least one of the frame parts at a central location adjacent to the translatory guide.

Such head-clamp mounting section may comprise a head-clamp that receives and holds a patient's head in place with respect to the patient table, or may at least comprise an interface for connecting to such head-clamp. Thus, the inventive mounting frame also serves as a support structure for a head-clamp. As the head-clamp connects to the mounting frame with only the head-clamp mounting section as an intermediate structure, the relative position of a patient's head and the robotic system is maintained in a most reliable manner for the benefit of high-precision craniotomy.

The head-clamp mounting section may be formed in the same manner and/or from the same material as the first and the second frame part of the mounting frame, particularly from a radiolucent material. Further, the head-clamp mounting section may connect to the mounting frame at a position underneath the patient table such that no or almost no “overhang” of the patient's head beyond the front edge of the patient table occurs.

In the following, the invention is described with reference to the appended Figures which give background explanations and represent specific embodiments of the invention. The scope of the invention is however not limited to the specific features disclosed in the context of the Figures, wherein

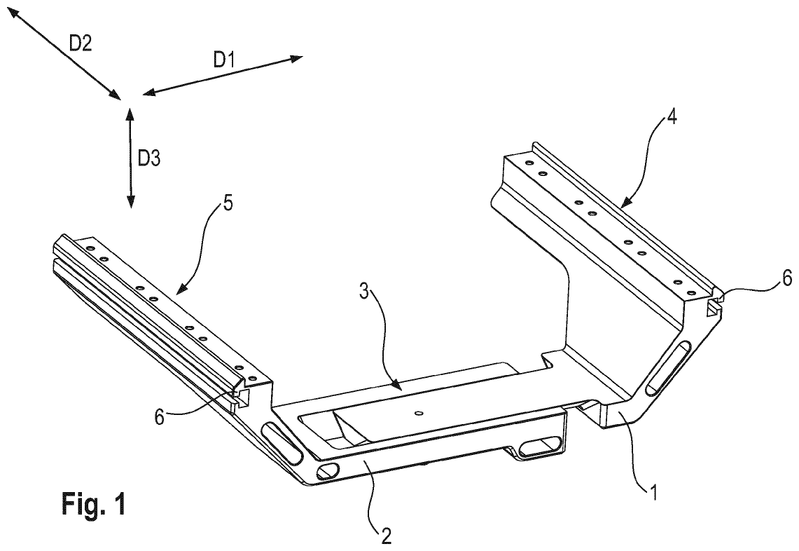

FIG. 1 shows the first and the second frame part as basic components of the inventive mounting frame;

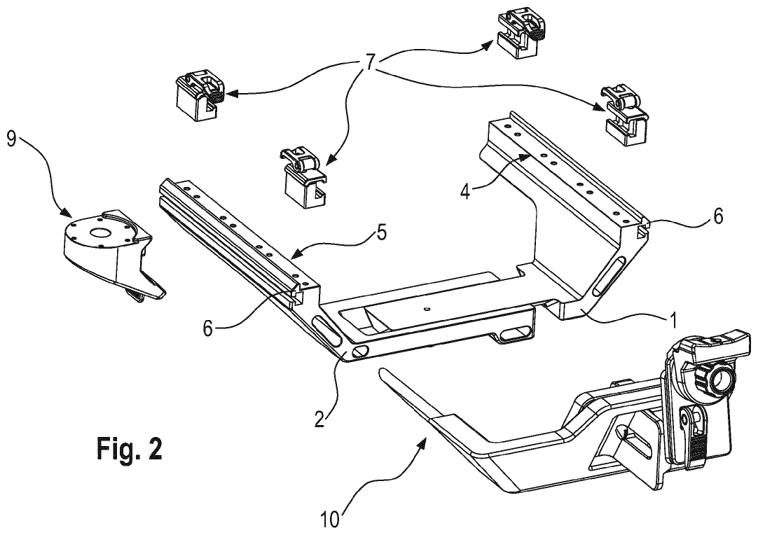

FIG. 2 shows the mounting frame of FIG. 1 with additional components of the mounting frame;

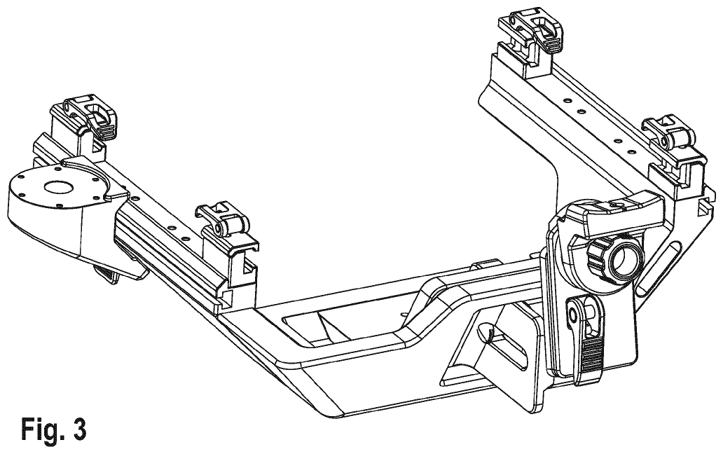

FIG. 3 shows the mounting frame of FIG. 2 in an assembled state;

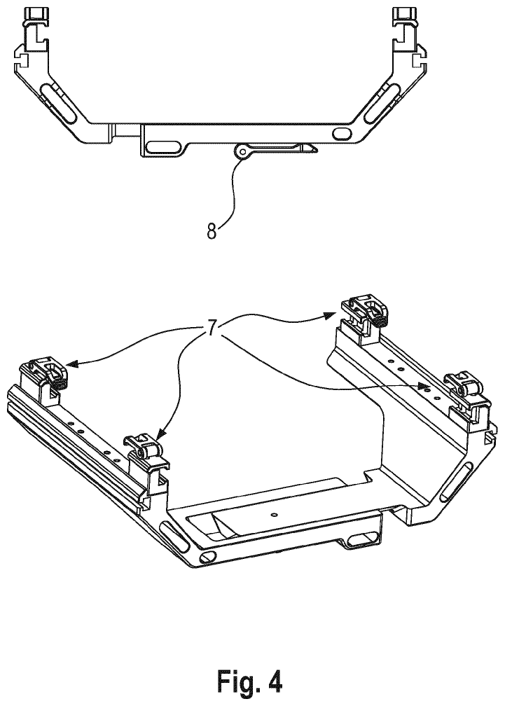

FIG. 4 shows a first embodiment of the mounting frame adapted to connect to patient table side-rails;

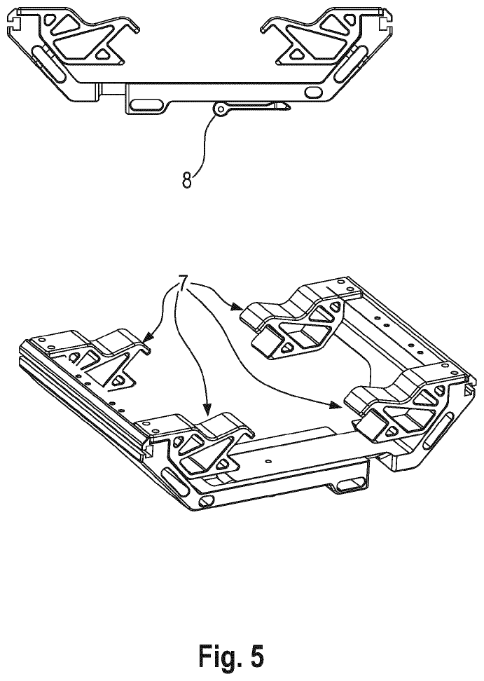

FIG. 5 shows a second embodiment of the mounting frame adapted to connect directly to a patient table frame;

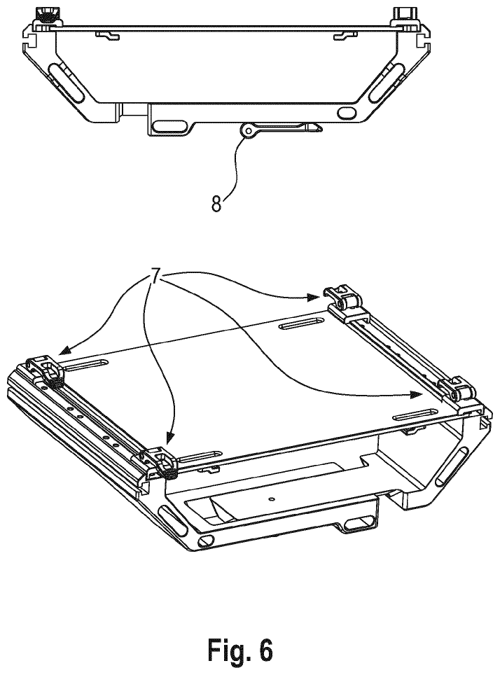

FIG. 6 shows a third embodiment of the mounting frame adapted to connect to an auxiliary support plate that rests on the tabletop of a patient table.

### DESCRIPTION OF EMBODIMENTS

FIG. 1 shows the basic part of the inventive mounting frame, namely a first frame part 1 and a second frame part 2 which directly connect each other in a translatory guide 3 that enables the two frame parts 1, 2 to move with respect to each other in a first direction D1. The translatory guide 3 is formed by a wedge shaped extension of the first frame part 1 and a correspondingly shaped recess within the second frame part 2. As soon as the frame parts 1 and 2 are pressed against each other in a direction D3 by a locking mechanism 8 comprising a lever (cf. FIGS. 4 to 6), this wedge shape increases the frictional force between the frame parts 1 and 2 which reliably holds the frame parts 1 and 2 at a desired position. At both sides of the translatory guide 3, the frame parts 1 and 2 extend in a direction D2, which sections are herein referred to as table engagement sections 4 and 5. In the shown example these engagement sections 4 and 5 include discrete fixation recesses via which engagement members 7 (cf. FIGS. 2 and 4 to 6) may be attached to the engagement sections 4 and 5 of the respective frame parts 1 and 2.

Further, both of the engagement sections 4 and 5 include a mounting section 6 represented by a rail which extends in the direction D2 and is adapted to receive a mounting adaptor 9 (cf. FIG. 2). Both of the frame parts 1 and 2 include recesses for receiving correspondingly shaped extensions of a head-clamp mounting section 10 (cf. FIG. 2) at their front faces.

FIG. 2 shows multiple components which are adapted to connect to the inventive mounting frame. FIG. 2 also shows four engagement members 7 which are configured to engage side-rails of a patient table. Each one of the engagement members 7 comprises a lever-activated clamping mechanism adapted to fix the respective engagement member 7 to a patient table side-rail.

FIG. 2 further shows a mounting adaptor 9 adapted to connect between a patient table and a robotic system (such as Cirq®, a product of Brainlab AG, Germany). In the shown example, the mounting adaptor 9 can be mounted to either one of the mounting sections 6 of the first or the second frame part 1, 2, respectively. Thus, the robotic system held by the mounting adaptor 9 can be either positioned left or right to the patient's head that is held via the head clamp mounting section 10 at a central location between the table engagement sections 4 and 5 and the mounting sections 6 thereof.

FIG. 3 shows the mounting frame of FIG. 2 in an assembled state.

As becomes apparent from FIGS. 4 to 6, various types of engagement members 7 may connect to the frame parts 1 and 2. Thus, the inventive mounting frame can attach to various types of patient tables having different geometries and dimensions, as long as suitably formed engagement members 7 are provided.

1. A patient table mounting frame comprising:
a first frame part; and
a second frame part,
wherein the first and second frame parts are adapted to receive an associated patient table between the first and second frame parts,
wherein the first and second frame parts are mutually connected with each other along a translatory guide that that couples the first and second frame parts to each other and enables the first frame part and the second frame part to move with respect to each other in a first direction,
wherein the first frame part and the second frame part each comprise a table engagement section adapted to releasably connect to an associated patient table or parts thereof,
wherein at least one of the first frame part and/or the second frame part includes a mounting section adapted to provide a releasable connection to a correspondingly formed associated mounting adaptor.

2. The patient table support frame according to claim 1, wherein the table engagement sections of the first frame part and the second frame part extend in a second direction transversal, and that is perpendicular to the first direction and/or wherein the table engagement sections of the first frame part and the second frame part extend parallel to each other.

3. The patient table support frame according to claim 1, wherein the table engagement sections of the first frame part and the second frame part are adapted to connect to the patient table or parts thereof over a predefined distance, particularly over at least 10 cm, specifically over at least 20 cm, more specifically over at least 30 cm.

4. The patient table support frame according to claim 1, wherein the table engagement sections of the first frame part and the second frame part each include one or more engagement members adapted to releasably engage at least one of:
a side-rail of the patient table which particularly extends laterally of the tabletop of the patient table;
a frame of the patient table;
an auxiliary support plate adapted to rest on the upper surface of the tabletop of the patient table;
particularly wherein at least one of the table engagement sections of the first frame part and the second frame part includes an engagement rail enabling the one or more engagement members to connect to a respective engagement section at a varying location along the engagement rail.

5. The patient table support frame according to claim 4, wherein one or more of the engagement members are adapted to releasably connect to the patient table or parts thereof via a friction fit and/or a positive fit established between the one or more engagement members and the patient table or parts thereof, wherein one or more of the engagement members include a clamping mechanism.

6. The patient table support frame according to claim 1, further comprising a locking mechanism adapted to selectively fix the relative position of the first frame part and the second frame part, wherein the locking mechanism selectively fixes the relative position of the first frame part and the second frame part via a friction or positive fit established between the first frame part and the second frame part.

7. The patient table support frame according to claim 1, further comprising a drive mechanism adapted to move the first frame part and the second frame part with respect to each other in the first direction, wherein the table engagement sections are adapted to releasably connect to the patient table or parts thereof by being pressed against the patient table or parts thereof via the drive mechanism and/or wherein the drive mechanism is manually activated.

8. The patient table support frame according to claim 1, wherein at least one of the first and/or second frame parts comprises a cross-sectional extension, and the other of the frame parts comprises a correspondingly shaped cross-sectional recess.

9. The patient table support frame according to claim 1, wherein the patient table support frame has a C-shaped geometry with the table engagement sections of the first frame part and the second frame part being spaced apart and extending substantially parallel to each other, and the translatory guide extending between respective ends of the engagement sections.

10. The patient table support frame according to claim 1, wherein an overall cross-sectional shape of the patient table support frame tapers downwards in a third direction perpendicular to the first direction and the second direction.

11. The patient table support frame according to claim 1, further comprising at least one mounting adaptor adapted to selectively connect to the mounting section of either one of the first frame part and/or the second frame part.

12. The patient table support frame according to claim 1, wherein the mounting section comprises an engagement rail enabling at least one mounting adaptor to connect to a respective mounting section at a varying location along the engagement rail.

13. The patient table support frame according to claim 1, wherein at least one mounting adaptor is configured to form part of or to connect to an associated automatic or semi-automatic medical robot or an associated articulated support arm.

14. The patient table support frame according to claim 1, wherein at least one mounting adaptor formed as a rail extending laterally to and at a distance from one of the table engagement sections.

15. The patient table support frame according to claim 1, further comprising:
a head clamp mounting section adapted to releasably connect to at least one of the first frame part and/or the second frame part at a central location adjacent to the translatory guide.

16. The patient table support frame according to claim 1, wherein at least one of the first and/or second frame parts comprises a tapered cross-sectional extension, and the other of the at least one of the first and/or second frame parts comprises a correspondingly shaped cross-sectional recess.
